(19) United States
(12) Patent Application Publication
(10) Pub. No.: US 2025/0169978 A1
(43) Pub. Date: May 29, 2025

(54) NON-INVASIVE ORTHOSIS FOR IMMOBILIZING THE CERVICAL SPINE

(71) Applicant: Duke University, Durham, NC (US)

(72) Inventors: Eli Johnson, Durham, NC (US); Amisha Gandhi, Durham, NC (US); Shruthee Sankarlinkam, Durham, NC (US); Jianing Chen, Durham, NC (US); Garima Iyer, Durham, NC (US); Lauren Lederer, Durham, NC (US); Jiselle Havas, Durham, NC (US); Rebecca Hogewood, Durham, NC (US); Vanessa Tam, Durham, NC (US); Bo Shaffer, Durham, NC (US)

(21) Appl. No.: 18/962,035

(22) Filed: Nov. 27, 2024

Related U.S. Application Data
(60) Provisional application No. 63/602,730, filed on Nov. 27, 2023.

(51) Int. Cl. A61F 5/058; A61F 5/37

(52) U.S. Cl. CPC A61F 5/05891; A61F 5/3769

(57) ABSTRACT

An orthosis is provided for immobilizing a cervical spine including: a forehead band; a back plate having a vertebral void, and a torso harness. The back plate is coupled to the forehead band. The torso harness includes a back plate stabilizer that stabilizes a position of the back plate relative to the cervical spine.

### CROSS-REFERENCE TO RELATED APPLICATION(S)

This application claims the benefit of U.S. Provisional Application Ser. No. 63/602,730, filed Nov. 27, 2023.

### BACKGROUND

A cervical spinal cord injury (SCI) is a serious, life-threatening emergency that can cause paralysis and loss of sensation throughout the entire body. Depending on the level and severity of a cervical spinal cord injury, it can affect major body functions like breathing and mobility from your neck down. The cervical spine includes the first seven vertebrae of the neck or the C1-C7 vertebrae.

Emergency Medical System (EMS) personnel often attempt to immobilize the cervical spine to prevent further injuries on the way to the hospital. This immobilization often involves use of a cervical collar, backboard, and headblocks. Significant concerns exist that many of these techniques fail to properly immobilize the cervical spine and/or immobilize movements of a patient, which results in increased injury.

### BRIEF SUMMARY

A non-invasive orthosis for isolating the cervical spine is presented. Advantageously, various implementations of the described non-invasive orthosis are able to be applied by EMS personnel, nurses, doctors, or anyone trained in applying the orthosis.

In position, the orthosis includes an adjustable back plate running from the back of the head down the spine, enabling it to be size adjusted for a patient. A forehead band and/or lateral brace set secure the head in an immobile position relative to the back plate so that up/down and side/side neck movements are inhibited. A torso vest stabilizes the position of the back plate so the back plate does not move relative to the spine. The back plate includes a vertebral void that enables X-rays and other images to be taken of the spine without removing the orthosis. In an example implementation, the vertebral void is a window in a central portion of the back plate approximately 20 mm wide, through which C1-C7 vertebrae are visible/exposed.

In some aspects, an orthosis is provided for immobilizing a cervical spine that includes: a forehead band; a back plate having a vertebral void, the back plate coupled to the forehead band; and a torso harness including a back plate stabilizer that stabilizes a position of the back plate relative to the cervical spine.

In some aspects, an orthosis is provided for immobilizing a cervical spine that includes: a forehead band; a back plate having a vertebral void, and a torso harness. The back plate is coupled to the forehead band. The torso harness includes a back plate stabilizer that stabilizes a position of the back plate relative to the cervical spine.

In some aspects, a method is provided for immobilizing a cervical spine of a wearer via an orthosis. The orthosis includes a forehead band, a back plate connected to the forehead band, and a torso harness. The method includes: securing the forehead band about a forehead of the wearer, wherein the forehead band is coupled to the back plate; positioning the back plate along a spine of the wearer such that a vertebral void in the back plate allows substantially unimpeded imaging of the cervical spine through the vertebral void; and placing a torso harness around a torso of the wearer. The torso harness stabilizes a position of the back plate relative to the cervical spine of the wearer. The securing, positioning, and placing prevent the wearer from performing up/down and side-to-side head movements.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1A is a structural diagram of an orthosis positioning back plate longitudinally along the spine, where the back plate is structurally connected to forehead band and torso harness.

FIG. 1B is a diagram of a human spine, which includes the upper spine or cervical region including the C1 to C7 vertebrae and cervical nerve roots.

FIG. 2A illustrates a front view of an embodiment of the orthosis of FIG. 1A.

FIG. 2B illustrates a back view of an embodiment of the orthosis of FIG. 1A.

FIG. 2C illustrates a back view of another embodiment of orthosis of FIG. 1A.

FIG. 2D shows a lateral brace hinged to a back plate consistent with the embodiment of FIG. 2C.

FIG. 3A illustrates a lateral element consistent with FIG. 2D.

FIG. 3B illustrates movement of segments at the hinges to emphasize the lateral element is adjustable to curve against and cradle a side of a head.

FIG. 3C illustrates a back plate having a vertebral void adjusted by extender to have a relatively short length.

FIG. 3D illustrates a back plate having a vertebral void adjusted by extender to have a relatively long length.

FIG. 3E shows one embodiment of a forehead band.

FIG. 4 shows a method for immobilizing a cervical spine of a wearer via an orthosis of FIG. 1.

### DETAILED DESCRIPTION

A non-invasive orthosis for isolating the cervical spine is presented. Advantageously, various implementations of the described non-invasive orthosis are able to be applied by EMS personnel, nurses, doctors, or anyone trained in applying the orthosis. In some cases, the described orthosis can be considered a cervical brace or truncal orthosis.

An orthosis for immobilizing the cervical spine facilitates the healing of damaged neck structures. In general, an orthosis stabilizes upper cervical spine injuries involving the C1-C2 vertebrae and provides support to the entire cervical spine, from C1-C7.

Conventional solutions for immobilizing the cervical spine include Halo vest immobilization (HVI) by PMT Corporation, which applies significant pressure on a patient's chest through a support extending from a front of the head to rest on the chest area of a patient. To illustrate, a 2005 publication by Majerck et. al. titled “Halo Vest Immobilization in the Elderly: a Death Sentence?” with PMID 16294074 found a high percentage of older HVI patients died from pneumonia and cardiac or respiratory arrest. Even for younger patients receiving HVI treatment, a constant downward pressure imposed on a chest region may be uncomfortable and can lead to complications.

Advantageously, various implementations of the non-invasive orthosis described herein do not require a structure that extends from a front of a wearer's head to a chest region of the wearer, avoiding the potential for the respiratory problems caused by pressure on the wearer's chest. Instead, sufficient support is provided along a wearer's back, which beneficially does not exert downward pressure on the chest. This is especially beneficial as individuals with injuries involving the C2 vertebrae. Those with C2 vertebrae injuries may experience difficulties breathing independently, often because of impaired function of the diaphragm, the main muscle responsible for respiration.

Further, the disclosed orthosis can protect the cervical spine without imposing a neck brace or similar structure about a neck of a wearer. Neck braces can be uncomfortable and many neck brace wearers have trouble breathing, which exacerbates respiratory problems known to exist with C2 injuries and with spinal immobilizing solutions like HVI, as mentioned.

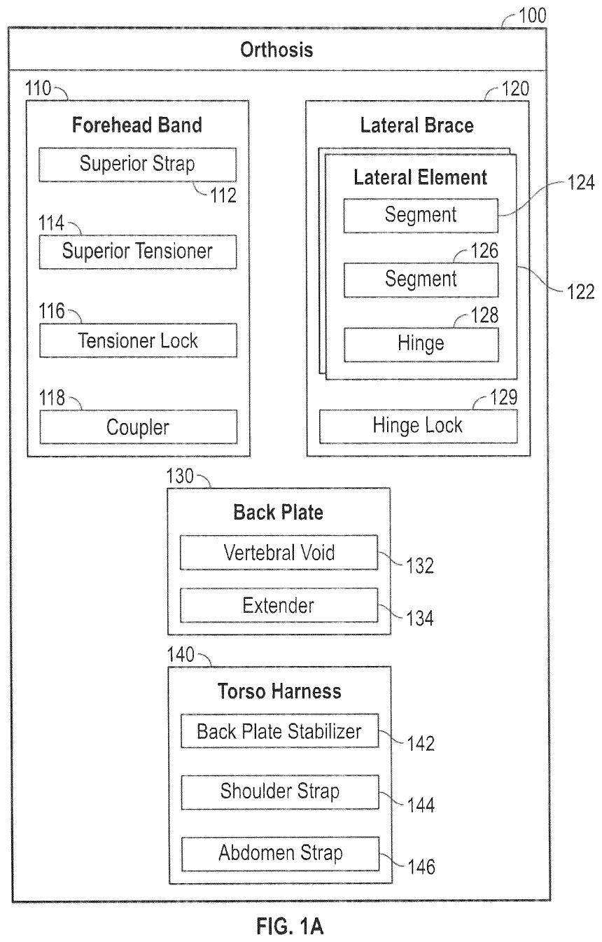

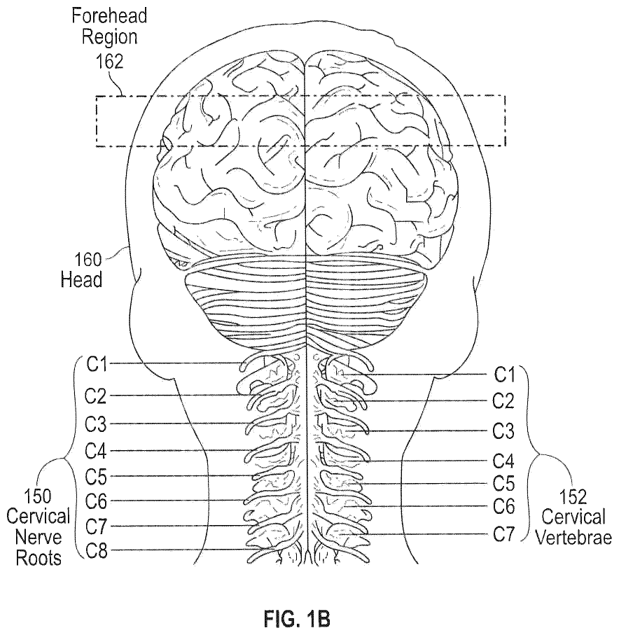

FIG. 1A is a structural diagram of an orthosis 100 positioning back plate 130 longitudinally along the spine, where the back plate 130 is structurally connected to forehead band 110 and torso harness 140. FIG. 1B is a diagram of a human spine, which includes the upper spine or cervical region including the C1 to C7 vertebrae 152 and cervical nerve roots 150. The upper cervical spine refers to the C1-2C vertebra. The lower cervical spine refers to the C3-C7 vertebrae.

When properly worn, the orthosis 100 immobilizes the head 160 to ensure fractures of the C1-C7 vertebrae 152 can heal. The immobilization is through posterior support along the cervical spine without components resting upon a chest region. Accordingly, orthosis 100 is a non-invasive alternative to a halo system (e.g. HVI) conventionally used for this purpose. Orthosis 100 is relatively simple to fit, which enables the orthosis to be utilized by EMS personnel transporting patients with potential neck injuries.

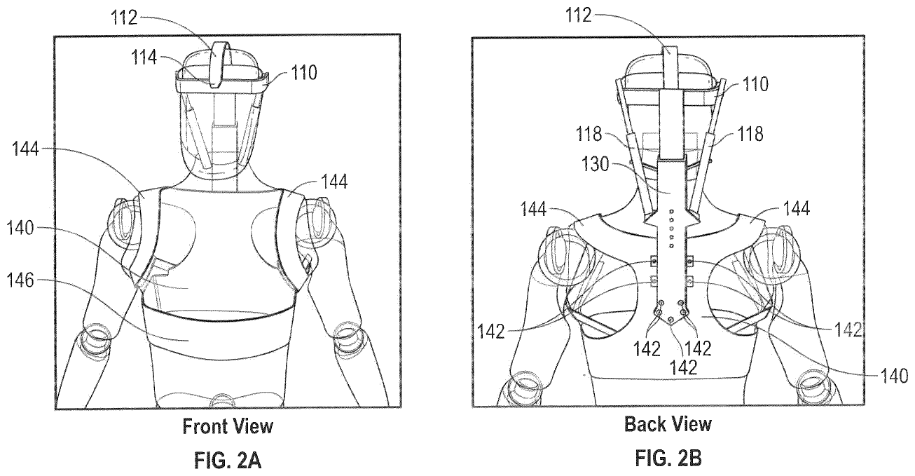

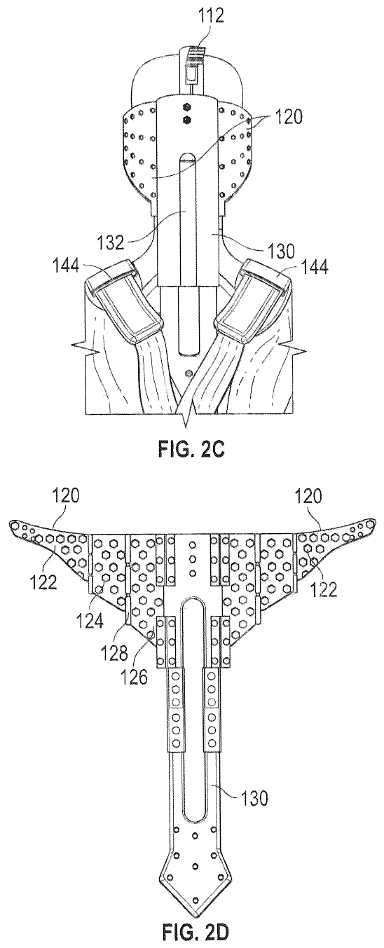

The forehead band 110 encircles the forehead region 162 in a non-invasive manner that fits snuggly enough that any movements of the forehead region 162 necessarily results in movement of the forehead band 110. For example, an interior of forehead band 110 can be glued using a skin adhesive to skin of the forehead of a wearer. A portion of back of a head can also be shaved to skin so that a rear of an interior of the forehead band 110 can be adhesively attached to this shaved portion of skin. Because the forehead band 110 is immobilized when orthosis 100 is worn, the head 160 is likewise immobilized, as are the C1-C7 vertebrae 152. (See FIGS. 2A and 2B for visual depiction of an embodiment of orthosis 100.)

A superior strap 112 can pass over a top of the head 160, where the superior strap 112 can support a position of the forehead band 110 about the head 160. Superior strap tensioner 114 adjusts a length of the superior strap 112 for fit. While both the forehead band 110 and the superior strap 112 are adjustable within a range, different size categorizes such as large, medium, and small can exist to account for different patient head sizes. Superior strap 112 can be especially beneficial when a wearer's head 160 is abnormally shaped. That is, some humans have cone shaped heads, which in absence of the superior strap 112 may cause upward slippage of the forehead band 110.

In some embodiments, tensioner lock 116 is provided to inhibit movements of the superior tensioner 114 after the orthosis 100 is properly fit to a patient. That is, medical professionals can engage the lock 116 after forehead band 110 is adjusted to properly cradle the head 160. A specialized unlocking device/key can be used with tensioner lock 116 to minimize adjustments by non-medical personnel.

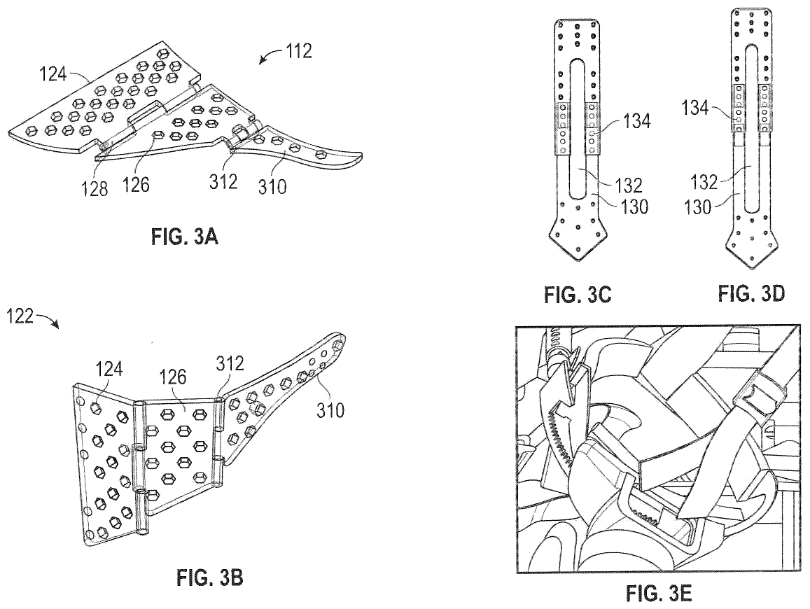

Coupler 118 securely attaches the forehead band 110 to the back plate 130. In some embodiments, the forehead band 110 can be formed from a plastic polymer, which is sufficiently flexible and adjustable to be fitted to a head 160 while being sufficiently rigid to inhibit movements (including up/down movements as well as side-to-side movements) of the head 160 when orthosis 100 is worn. Compressible material, such as foam or padding, can be used to enhance fit and comfort. FIG. 3E shows one embodiment of a forehead band 110.

In one embodiment, lateral brace 120 is utilized to stabilize a head 160. The lateral brace 120 can be implemented in conjunction with the forehead band 110 and/or as an alternative to the forehead band 110. In certain embodiments, the lateral brace 120 and respective lateral elements 122 are formed from a hard metal, such as steel or titanium. Other rigid materials providing sufficient support are contemplated. (See FIGS. 2C and 2D).

The lateral brace 120 includes two lateral elements 122 that are positioned on opposing sides of a head 160. Each lateral element 122 is adjustable laterally to be positioned snuggly against the surface of the head 160. In certain embodiments, a compressible foam (or other intervening material) can be coupled to an interior of the lateral elements 122, which will contact the sides of the head. In certain embodiments, once positioned about a head 160, front portions of the lateral elements 122 can be connected together by a joining segment. In some cases, the joining segment can be secured to a center of a forehead, for example, by adhesive. The joining segment, when used, results in the lateral element 122 forming a band about the head 160, which helps secure a position of the lateral elements 122.

Each of the lateral elements 122 can include a set of at least two segments 124 and 126, which are referred to as temporal segments as they pass along a temple region. Each segment 124, 126 of the lateral elements 122 can be connected via a hinge 128. In some embodiments, each lateral element 122 includes three or more segments 124, 126 connected by a hinge 128 set. For example, a three-segment lateral element 122 can include a third temporal segment 310 connected to the second temporal segment 126 via segment hinge 312 (See FIGS. 3A and 3B).

Each hinge 128 can have an associated hinge lock 129. Hinge lock 129 includes an open state (e.g., unlocked) allowing movement and a closed (e.g., locked) state inhibiting movement. In some embodiments, each lateral element 122 is hinged to the back plate 130, where that hinge also includes a lock with an open and closed state. (See FIG. 2C and FIG. 2D)

In some embodiments, the hinge 128 (as well as the hinge to the back plate 130) uses a screw having a least partial threading as a hinge pin. Tightening the screw with a tool, results in the segment hinge 128 being locked. Accordingly, the screw hinge pin along with screw locking arrangements may constitute hinge lock 129.

In some embodiments, a non-standard screw head can be used, which requires use of a specialized screw tip made available to medical personnel only. Other arrangements for hinge lock 129 are contemplated.

Back plate 130 is a substantially rigid structure extending from a lower portion of a head 160 to below the C7 vertebrae 152. Back plate 130 can be formed from a metal, such as steel or titanium. Other materials, such as basalt, stone, carbon fiber, tungsten, high impact plastics and polymers, can be used alone or in combination for back plate 130.

In some embodiments, a middle portion of the back plate 130 is substantially rectangular and shaped for comfort when extending longitudinally along a spine. An upper portion of the back plate 130 includes attachment points facilitating attachment to the forehead band 110, lateral brace 120, or both forehead band 110 and lateral brace 120. A lower portion of the back plate 130 can include attachment points facilitating attachment to the torso harness 140. (See FIGS. 2B and 2C)

The back plate 130 can include a vertebral void 132 in its middle portion between upper and lower regions, which include attachment points. The vertebral void 132 can be sufficiently wide, such as at least 20 mm, and sufficiently long to permit a window to the C1-C7 vertebrae 152. The vertebral void 132 allows x-rays and other imaging signals to pass through air or an effectively transparent region (for the imaging signals), which allows for imaging operations when the back plate 130 is worn. In certain embodiments, an upper width of the vertebral void 132 is restricted to minimize total width of the back plate 130, which is beneficial for patient comfort and practicality.

The back plate 130 includes an adjustment mechanism or extender 134 for changing a length of the back plate 130. The extender 134 permits a length of the back plate 130 to be adjusted to accommodate different sized wearers. In some implementations, three different sized back plates 130, such as small(S), medium (M), and large (L) along with the adjustments of each can reasonably accommodate divergent heights of a majority of the population. Adjustments of extender 134 ensure the vertebral void 132 is sufficiently sized to ensure imaging of the C1-C7 vertebrae 152 is substantially unimpeded. In one embodiment, a gear is integrated into the extender 134, such that turning the gear extends or contracts a length of the back plate 130. In another embodiment, a sequence of pin holes (e.g., at set intervals) are integrated for extender 134, where holes correspond to potential pin positions. Pins lock the length of back plate 130 by extending through aligned holes. In some embodiments, the extender 134 can be designed with a locking mechanism so it is only adjustable by trained medical personnel possessing a corresponding key to tool to unlock the locking mechanism. FIGS. 3C and 3D illustrate contemplated, non-limiting, arrangements for the back plate 130.

Torso harness 140 is a fabric, shaped plastic, or the other material designed to be worn about a torso. Torso harness 140 provides sufficient support to ensure a position of the back plate 130 relative to the spine is maintained. In some embodiments, different sizes, such as small(S), medium (M), and large (L) can be established for torso harness 140 to accommodate different sized torsos. Each sized torso harness 140 can also include fit adjustments, such as adjustments for shoulder strap 144 and abdomen strap 146.

The torso harness 140 includes a back plate stabilizer 142 that stabilizes the position of the back plate 130. In one embodiment, the back plate stabilizer 142 includes a series of rivets or other fixed coupler connecting a bottom portion of the back plate 130 to the torso harness 140. (See FIG. 2B).

In other embodiments, the back plate stabilizer 142 is detachably coupled to the back plate 130. For example, the torso harness 140 can include a rigid concavity mated to a lower portion of the back plate stabilizer 142 so that this portion fits into the concavity. A lock, latch, or other such securing component of the stabilizer 142 can ensure the lower portion of the back plate 130 is immobile relative to the torso harness 140 when coupled. The pentagon shape for the back plate 130 shown in FIG. 3C is one example of a shape designed to facilitate such a coupling with back plate stabilizer 142.

The shoulder strap 144 supports an upper portion of the torso harness 140 by fitting around the arms of a wearer in a manner similar to a backpack. The abdomen strap 146 supports a lower portion of the torso harness 140 by fitting around the abdomen of a wearer. Adjustments of straps 144 and 146 can be effectuated through use of an adjustable buckle, a hook-and-loop fastener, and the like.

In various implementations, micro-lattice padding can line rigid components of orthosis 100 for wearer comfort.

FIG. 2A illustrates a front view 200 of an embodiment of the orthosis 100 of FIG. 1A. Torso harness 140 is placed so that the shoulder straps 144 are about the shoulders and the abdomen strap 146 is placed about the abdomen. As shown, straps 144 and 146 use a hook-and-loop fastener. From the front, the forehead band 110 is visible with a superior strap 112 extending over a top of the wearer's head. Superior tensioner 114 is implemented as a knob that adjusts a length of the superior strap 112. As shown, torso harness 140 is formed using a fabric material. Forehead band 110 utilizes a plastic material having an interior lining.

From the front view, it is evident that no components of orthosis 100 rest upon a chest region as support is provided posterior to the back, along the spine by back plate 130. Further, a neck of the wearer is not encircled. Wearers of competing products, such as the Halo system, experience respiratory problems due to elements attached to the chest region that exert a downward pressure upon this chest region. Wearers of competing products are also known to experience respiratory problems due to elements that encircle the neck of the wearer, which makes breathing more difficult. Thus, orthosis 100 alleviates these problems present in competing products.

FIG. 2B illustrates a back view of an embodiment of the orthosis 100 of FIG. 1A. As shown, the back plate 130 extends from the head to below the C7 vertebrae. The embodiment shown does not have a vertebral void 132. The extender 134 is implemented using a pin that locks into a series of central holes. The coupler 118 includes two slanting rods slanting down from a forehead band 110 attachment point to an attachment point of the back plate 130. The back plate stabilizer 142 is implemented using a set of rivets or bolts that pass through the back plate 130 and the torso harness 140.

FIG. 2C illustrates a back view of another embodiment of orthosis 100 of FIG. 1A. The vertebral void 132 is shown through which unimpeded imaging of the cervical vertebrae occurs. Lateral brace 120 includes two lateral elements 122 positioned on opposing sides of a head. Superior strap 112 is directly connected to the back plate 130 as illustrated. In embodiments, a forehead band 110 (not shown for greater visibility of lateral brace 120) can be integrated with lateral brace 120 or can be positioned above lateral brace 120. Embodiments that include lateral brace 120 as an alternative to forehead band 110 are also contemplated.

FIG. 2D shows a lateral brace 120 hinged to a back plate 130 consistent with the embodiment of FIG. 2C. Two sets of hinges secure each lateral element 122 to the back plate 130. Each lateral element 122 includes three segments, each joined to an adjacent segment via a lockable hinge. The lateral brace 120 tapers upwardly so that it is thicker closer to the center of the back of the head and thinner closer to a front of the head. A wing shape for lateral brace 120 is shown, but others are contemplated.

FIG. 3A illustrates a lateral element 122 consistent with FIG. 2D. As shown, a first segment 124 is connected to a second segment 126 via hinge 128. Second segment 126 is connected to third segment 310 via hinge 312. Each of the hinges 128 and 312 can be implemented as locking hinges. The hinges 128 and 312 permit the segments to be pivoted inward towards the head, to secure the head in place. Segments 124, 126, and 310 can be substantially flat or may be implemented with a curvature consistent with a head curvature. Each segment 124, 126, and 310 can be considered a temporal segment, the lateral element 122 is positioned along a temple of a wearer.

FIG. 3B illustrates movement of segments 124, 126, and 310 at the hinges 128, 312 to emphasize the lateral element 122 is adjustable to curve against and cradle a side of a head.

FIG. 3C illustrates a back plate 130 having a vertebral void 132 adjusted by extender 134 to have a relatively short length.

FIG. 3D illustrates a back plate 130 having a vertebral void 132 adjusted by extender 134 to have a relatively long length.

FIG. 3E shows an image of an adjustable forehead band.

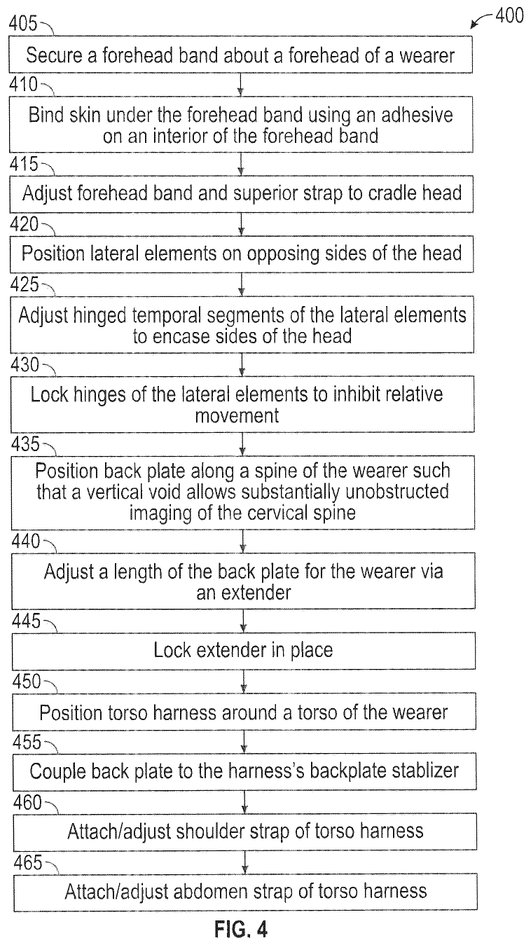

FIG. 4 shows a method 400 for immobilizing a cervical spine of a wearer via an orthosis 100 of FIG. 1. In step 405, a forehead band can be secured about a forehead of the wearer. In step 410, skin can be bound to the forehead band using an adhesive placed on an interior of the forehead band. In step 415, the forehead band as well as a superior strap extending over a top of a head are adjusted to cradle the head.

In some embodiments of the method 400, a lateral brace will be integrated into the orthosis 100 causing steps 420-430 to be performed. An implementation lacking a lateral brace can skip to step 435. In step 420, lateral elements of the lateral brace can be positioned on opposing sides of a head. In step 425, hinged temporal segments of each lateral element are adjusted to encase sides of the head. In step 430, the hinges are locked to inhibit relative movement of the position-adjusted temporal segments.

In step 425, the back plate can be positioned longitudinally along the spine of a wearer. This positioning can ensure a vertebral void in the back plate is present behind the C1-C7 vertebrae. This ensures unobstructed imaging, such as X-ray imaging, is possible without removing the back plate. In step 440, a length of the back plate is adjusted for the wearer via an extender. The extender is locked into place in step 445.

In step 450, a torso harness is positioned around a torso of the wearer. In step 455, the back plate is coupled to the torso harness's back plate stabilizer. Step 455 is needed in embodiment where the back plate decouples from the torso harness, which can be a beneficial relationship for orthosis sizing and storage. In step 460, one or more shoulder straps can be adjusted or otherwise attached to the torso harness. In step 465 one or more abdomen straps can be adjusted or otherwise attached to the torso harness.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

What is claimed is:

1. An orthosis for immobilizing a cervical spine comprising:
a forehead band;
a back plate having a vertebral void, the back plate coupled to the forehead band; and
a torso harness comprising a back plate stabilizer that stabilizes a position of the back plate relative to the cervical spine.

2. The orthosis of claim 1, wherein the forehead band further comprises:
a superior strap; and
a superior strap tensioner for adjusting a length of the superior strap.

3. The orthosis of claim 1, wherein the back plate further comprises:
an extender for changing a length of the back plate.

4. The orthosis of claim 1, wherein the torso harness further comprises:
a shoulder strap; and
an abdomen strap.

5. The orthosis of claim 1, further comprising:
a lateral brace hinged to the back plate.

6. The orthosis of claim 5, wherein the lateral brace comprises:
two lateral elements positioned on opposing sides of a head.

7. The orthosis of claim 6, wherein each of the two lateral elements comprise:
a first temporal segment;
a second temporal segment; and
a segment hinge joining the first temporal segment and the second temporal segment.

8. The orthosis of claim 7, wherein the segment hinge and a hinge set between the lateral brace and the back plate comprise:
a hinge lock having an open state allowing movement and a closed state inhibiting movement.

9. The orthosis of claim 1, wherein the vertebral void permits unimpeded imaging of cervical vertebrae when the orthosis is worn.

10. The orthosis of claim 1, wherein the forehead band cradles a head of a wearer, wherein the torso harness is worn about a torso of the wearer, wherein the back plate in arrangement with the forehead band and the torso harness prevents the wearer from performing up/down and side-to-side head movements.

11. The orthosis of claim 1, wherein the orthosis immobilizes the cervical spine through posterior support along the cervical spine without components resting upon a chest region, which alleviates respiratory problems experienced by wearers of competing products having elements attached to the chest region that exert a downward pressure upon the chest region.

12. The orthosis of claim 1, wherein the orthosis immobilizes the cervical spine through posterior support along the cervical spine without encircling a neck of a wearer, which alleviates respiratory problems experienced by wearers of competing products having elements that encircle the neck of the wearer.

13. An orthosis for immobilizing a cervical spine comprising:
a lateral brace comprising two lateral elements positioned on opposing sides of a head;
a back plate, the back plate being hinged to each of the two lateral elements; and
a torso harness comprising a back plate stabilizer that stabilizes a position of the back plate relative to the cervical spine.

14. The orthosis of claim 13, the back plate comprising:
a vertebral void at least 20 mm wide and of sufficient length to extend from C1 to C7 vertebrae of the cervical spine.

15. The orthosis of claim 13, wherein each of the two lateral elements comprise:
a first temporal segment;
a second temporal segment; and
a segment hinge joining the first temporal segment and the second temporal segment.

16. A method for immobilizing a cervical spine of a wearer via an orthosis, said orthosis comprising a forehead band, a back plate connected to the forehead band, and a torso harness, said method comprising:
securing the forehead band about a forehead of the wearer, wherein the forehead band is coupled to the back plate;
positioning the back plate along a spine of the wearer such that a vertebral void in the back plate allows substantially unimpeded imaging of the cervical spine through the vertebral void; and
placing a torso harness around a torso of the wearer, wherein the torso harness stabilizes a position of the back plate relative to the cervical spine of the wearer, wherein the securing, positioning, and placing prevent the wearer from performing up/down and side-to-side head movements.

17. The method of claim 16, further comprising:
adjusting a length of the back plate for the wearer via an extender; and
locking the extender of the back plate.

18. The method of claim 16, further comprising:
gluing skin under the forehead band using an adhesive on an interior of the forehead band.

19. The method of claim 16, wherein the orthosis comprises two lateral elements, the method further comprising:
positioning the lateral elements on opposing sides of a head of the wearer; and
locking a hinge set between the lateral elements and the back plate to inhibit movement.

20. The method of claim 16, wherein the orthosis comprises two lateral elements, wherein each of the two lateral elements comprise:
a first temporal segment;
a second temporal segment; and
a segment hinge joining the first temporal segment and the second temporal segment, the method further comprising:
positioning the lateral elements on opposing sides of a head of the wearer;
adjusting the first temporal segment and the second temporal segment of each of the lateral elements to encase sides of the head; and
locking the segment hinges to inhibit movement of the first and second temporal segments relative to each other.
